(19) United States
(12) Patent Application Publication
(10) Pub. No.: US 2022/0392578 A1
(43) Pub. Date: Dec. 8, 2022

(54) APPARATUS AND METHOD FOR GENOME SEQUENCE ALIGNMENT ACCELERATION

(71) Applicant: ELECTRONICS AND TELECOMMUNICATIONS RESEARCH INSTITUTE, Daejeon (KR)

(72) Inventors: Chang-Dae KIM, Daejeon (KR); Kwang-Won KOH, Daejeon (KR); Kang-Ho KIM, Daejeon (KR); Tae-Hoon KIM, Daejeon (KR)

(21) Appl. No.: 17/832,252

(22) Filed: Jun. 3, 2022

(30) Foreign Application Priority Data
Jun. 4, 2021 (KR) 10-2021-0072711
Apr. 19, 2022 (KR) 10-2022-0048190

(51) Int. Cl. G16B 30/10

(52) U.S. Cl. CPC G16B 30/10

(57) ABSTRACT

Disclosed herein are an apparatus and method for accelerating genome sequence alignment. The method may include loading an essential index for a reference genome into memory, loading an additional index corresponding to the amount of available memory into memory, reading a target nucleotide sequence for which genome sequence alignment is to be performed, checking whether an exact match of the target nucleotide sequence is present in the reference genome based on the additional index, and generating a result of alignment of the target nucleotide sequence using the location of the exact match of the target nucleotide sequence in the reference genome when an exact match is found.

### CROSS REFERENCE TO RELATED APPLICATIONS

This application claims the benefit of Korean Patent Application No. 10-2021-0072711, filed Jun. 4, 2021, and No. 10-2022-0048190, filed Apr. 19, 2022, which are hereby incorporated by reference in their entireties into this application.

### BACKGROUND OF THE INVENTION

### 1. Technical Field

The disclosed embodiment relates to technology for genome sequence alignment acceleration.

### 2. Description of the Related Art

Genome sequence alignment refers to determination of the location of a short nucleotide sequence read from a human or another organism in a reference genome that consists of the entire genome of the human or organism. Here, because all genomes are different and because an error may occur when reading a nucleotide sequence, the location of the sequence that is most similar to the target nucleotide sequence is searched for and identified in the reference genome in consideration of insertions, deletions, and mutations of the nucleotide sequence.

It is very costly to map the entire genome of a human or an organism of a specific species. However, the use of genome sequence alignment makes it possible to construct the entire genome merely by reading a large number of short nucleotide sequences from a human or an organism, whereby the entire genome can be analyzed at low cost. Also, through this, the cause of diseases resulting from genetic mutation or variation may be easily detected.

Genome sequence alignment described above is possible due to the high similarity between genomes. That is, because the genome of two different humans is mostly the same, when a short nucleotide sequence is given, the part that is most similar thereto is searched for in a reference genome, and the location of the found part may be inferred to be the location of the short nucleotide sequence. The difference in genomes between people is 0.1% on average, and there is research saying that, for a nucleotide sequence having a length of 100, 90% or more thereof exactly matches a reference genome. However, this figure was acquired without consideration of errors introduced by sequencing machines, and when error is considered in the same research, the actual match rate was reported to be 67.6%.

Meanwhile, as mechanisms commonly used for genome sequence alignment, there are a Burrows-Wheeler Transform (BWT) algorithm and a Ferragina-Manzini (FM) index structure. Using such a mechanism, the location of a short string in a long string can be efficiently searched for. This mechanism is performed in such a way that locations of the first character of a short string are searched for in a long string, and among the found locations, locations at which the first character is followed by the second character of the short string are then searched for.

Also, various kinds of hardware devices (FPGA, ASIC, etc.) and software technologies for fast processing of genome sequence alignment are currently available. Using hardware devices may quicken a specific step of sequence alignment. However, the use of a hardware device requires the device itself and special equipment in which the device can be installed, and only the specific step to which the corresponding hardware is applicable is accelerated. Also, the device may affect the accuracy of sequence alignment.

Software technologies have advantages in that they can be immediately applied to general computers. However, software technology requiring a large amount of memory may be difficult to apply to already constructed systems. For example, when a hash table is used in order to quickly find an exact match, tens to hundreds of gigabytes of memory are additionally required, so it is difficult to execute the software on a general computer.

In a computer system, memory is a major determinant as to whether it is possible to execute a program. Unless the amount of memory required by a program is secured, the program cannot be executed. Therefore, when a computer system is constructed, it is required to equip the same with the expected maximum amount of memory. Therefore, most of the time, some of the memory is not used, but remains idle.

### SUMMARY OF THE INVENTION

An object of the disclosed embodiment is to improve the speed of genome sequence alignment depending on the available memory capacity.

Another object of the disclosed embodiment is to make use of available memory in a system, thereby improving the speed of genome sequence alignment without special hardware.

An apparatus for accelerating genome sequence alignment according to an embodiment includes memory in which at least one program is recorded and a processor for executing the program. The program may perform loading an essential index for a reference genome into memory, loading an additional index corresponding to the amount of available memory into memory, reading a target nucleotide sequence for which genome sequence alignment is to be performed, checking whether an exact match of the target nucleotide sequence is present in the reference genome based on the additional index, and generating the result of alignment of the target nucleotide sequence using the location of the exact match in the reference genome when the exact match is found.

Here, when loading the additional index into memory, the program may use available memory, the amount of which is calculated by subtracting the size of the essential index from a total amount of memory to be used for indexes for genome sequence alignment, in order to load the additional index.

Here, when loading the additional index into memory, if the additional index comprises two or more additional indexes, the program may sequentially load the additional indexes, and the order in which the additional indexes are loaded may be determined based on the effect of each of the additional indexes on genome sequence alignment performance.

Here, when loading the additional index into memory, the program may load all or part of the additional index depending on whether the amount of available memory is equal to or greater than the size of the additional index to be loaded, and when part of the additional index is loaded, the program may preferentially load the essential part of the additional index.

Here, the additional index may include a first index that is used when checking whether the exact match of the target nucleotide sequence is present in the reference genome is performed, and the first index may include a seed table configured with hash entries corresponding to respective seeds having a predetermined length, which are extracted from the reference genome, and a multi-location table in which two or more locations of an identical seed in the reference genome are collectively mapped to a single index.

Here, the hash entry may include information about the location of a seed in the reference genome, information about whether the hash entry has a hash collision, an index number of the next hash entry having the same hash value as the hash entry, and information about an index in the multi-location table.

Here, when checking whether the exact match of the target nucleotide sequence is present in the reference genome based on the additional index, the program may perform calculating the hash value of the target nucleotide sequence; searching for a hash entry corresponding to the hash value when the hash value is less than the number of loaded hash entries of the seed table; extracting, when the hash entry corresponding to the hash value is found and when the found entry is not an entry having a hash collision, a seed from the reference genome using location information stored in the found entry; checking whether the extracted seed matches the target nucleotide sequence; and searching, when the extracted seed is determined to match the target nucleotide sequence, the multi-location table for all exact matches of the target nucleotide sequence in the reference genome.

Here, when checking whether the extracted seed matches the target nucleotide sequence is performed, if it is determined that the extracted seed does not match the target nucleotide sequence, the program may search for an entry corresponding to the next value of the hash entry in the seed table and further perform checking whether a seed of the found entry matches the target nucleotide sequence.

Here, when the exact match of the target nucleotide sequence is not found in the reference genome, the program may perform finding a maximal exact match between the target nucleotide sequence and the reference genome based on the essential index, measuring the degree of matching between the target nucleotide sequence and the maximal exact match found in the reference genome, and generating a result indicating the degree of matching, and when finding the maximal exact match is performed, the program may accelerate an initial step of finding the maximal exact match based on a second index of the additional index.

A method for accelerating genome sequence alignment according to an embodiment may include loading an essential index for a reference genome into memory, loading an additional index corresponding to the amount of available memory into memory, reading a target nucleotide sequence for which genome sequence alignment is to be performed, checking whether an exact match of the target nucleotide sequence is present in the reference genome based on the additional index, and generating a result of alignment of the target nucleotide sequence using the location of the exact match in the reference genome when the exact match is found.

Here, loading the additional index into memory may comprise loading all or part of the additional index depending on whether the amount of available memory is equal to or greater than the size of the additional index to be loaded, and when part of the additional index is loaded, the essential part of the additional index may be preferentially loaded.

Here, the additional index may include a first index that is used when checking whether the exact match of the target nucleotide sequence is present in the reference genome is performed, and the first index may include a seed table configured with hash entries corresponding to respective seeds having a predetermined length, which are extracted from the reference genome, and a multi-location table in which two or more locations of an identical seed in the reference genome are collectively mapped to a single index.

Here, the hash entry may include information about the location of a seed in the reference genome, information about whether the hash entry has a hash collision, an index number of the next hash entry having the same hash value as the hash entry, and information about an index in the multi-location table.

Here, checking whether the exact match of the target nucleotide sequence is present in the reference genome based on the additional index may include calculating the hash value of the target nucleotide sequence; searching for a hash entry corresponding to the hash value when the hash value is less than the number of loaded hash entries of the seed table; extracting, when the hash entry corresponding to the hash value is found and when the found entry is not an entry having a hash collision, a seed from the reference genome using location information stored in the found entry; checking whether the extracted seed matches the target nucleotide sequence; and searching, when the extracted seed is determined to match the target nucleotide sequence, the multi-location table for all exact matches of the target nucleotide sequence in the reference genome.

The method may further include, when it is determined that the extracted seed does not match the target nucleotide sequence as the result of checking whether the extracted seed matches the target nucleotide sequence, searching for an entry corresponding to the next value of the hash entry in the seed table and checking whether a seed of the found entry matches the target nucleotide sequence.

The method may further include, when the exact match of the target nucleotide sequence is not found in the reference genome, finding a maximal exact match between the target nucleotide sequence and the reference genome based on the essential index, measuring the degree of matching between the target nucleotide sequence and the maximal exact match found in the reference genome, and generating a result indicating the degree of matching. When finding the maximal exact match is performed, an initial step of finding the maximal exact match may be accelerated based on a second index of the additional index.

A method for accelerating genome sequence alignment according to an embodiment may include loading an essential index for a reference genome into memory, loading an additional index corresponding to the amount of available memory into memory, reading a target nucleotide sequence for which genome sequence alignment is to be performed, checking whether an exact match of the target nucleotide sequence is present in the reference genome based on a first index of the additional index, generating a result of alignment of the target nucleotide sequence using the location of the exact match in the reference genome when the exact match is found, finding a maximal exact match between the target nucleotide sequence and the reference genome based on the essential index when the exact match of the target nucleotide sequence is not found, measuring the degree of matching between the target nucleotide sequence and the maximal exact match found in the reference genome, and generating a result indicating the degree of matching. When finding the maximal exact match is performed, an initial step of finding the maximal exact match may be accelerated based on a second index of the additional index.

Here, the first index may include a seed table configured with hash entries corresponding to respective seeds having a predetermined length, which are extracted from the reference genome, and a multi-location table in which two or more locations of an identical seed in the reference genome are collectively mapped to a single index, and the hash entry may include information about the location of a seed in the reference genome, information about whether the hash entry has a hash collision, an index number of the next hash entry having the same hash value as the hash entry, and information about an index in the multi-location table.

Here, checking whether the exact match of the target nucleotide sequence is present in the reference genome based on the first index may include calculating the hash value of the target nucleotide sequence; searching for a hash entry corresponding to the hash value when the hash value is less than the number of loaded hash entries of the seed table; extracting, when the hash entry corresponding to the hash value is found and when the found entry is not an entry having a hash collision, a seed from the reference genome using location information stored in the found entry; checking whether the extracted seed matches the target nucleotide sequence; and searching, when the extracted seed is determined to match the target nucleotide sequence, the multi-location table for all exact matches of the target nucleotide sequence in the reference genome.

The method may further include, when it is determined that the extracted seed does not match the target nucleotide sequence as a result of checking whether the extracted seed matches the target nucleotide sequence, searching for an entry corresponding to the next value of the hash entry in the seed table and checking whether a seed of the found entry matches the target nucleotide sequence.

### BRIEF DESCRIPTION OF THE DRAWINGS

The above and other objects, features, and advantages of the present invention will be more clearly understood from the following detailed description taken in conjunction with the accompanying drawings, in which:

FIG. 1 is a flowchart for explaining a method for accelerating genome sequence alignment according to an embodiment;

FIG. 2 is a flowchart for explaining in detail a step of loading an additional index into memory according to an embodiment;

FIG. 3 is an exemplary view of a first index for quickly searching for an exact match of a target nucleotide sequence according to an embodiment;

FIG. 4 is a flowchart for explaining in detail a step of quickly checking whether an exact match of a target nucleotide sequence is present according to an embodiment;

FIG. 5 is an experimental result of implementation of an embodiment in BWA-MEM2, which is a genome-sequencing program; and

FIG. 6 is a view illustrating a computer system configuration according to an embodiment.

### DESCRIPTION OF THE PREFERRED EMBODIMENTS

The advantages and features of the present invention and methods of achieving the same will be apparent from the exemplary embodiments to be described below in more detail with reference to the accompanying drawings. However, it should be noted that the present invention is not limited to the following exemplary embodiments, and may be implemented in various forms. Accordingly, the exemplary embodiments are provided only to disclose the present invention and to let those skilled in the art know the category of the present invention, and the present invention is to be defined based only on the claims. The same reference numerals or the same reference designators denote the same elements throughout the specification.

It will be understood that, although the terms “first,” “second,” etc. may be used herein to describe various elements, these elements are not intended to be limited by these terms. These terms are only used to distinguish one element from another element. For example, a first element discussed below could be referred to as a second element without departing from the technical spirit of the present invention.

The terms used herein are for the purpose of describing particular embodiments only, and are not intended to limit the present invention. As used herein, the singular forms are intended to include the plural forms as well, unless the context clearly indicates otherwise. It will be further understood that the terms “comprises,” “comprising,”, “includes” and/or “including,” when used herein, specify the presence of stated features, integers, steps, operations, elements, and/or components, but do not preclude the presence or addition of one or more other features, integers, steps, operations, elements, components, and/or groups thereof.

Unless differently defined, all terms used herein, including technical or scientific terms, have the same meanings as terms generally understood by those skilled in the art to which the present invention pertains. Terms identical to those defined in generally used dictionaries should be interpreted as having meanings identical to contextual meanings of the related art, and are not to be interpreted as having ideal or excessively formal meanings unless they are definitively defined in the present specification.

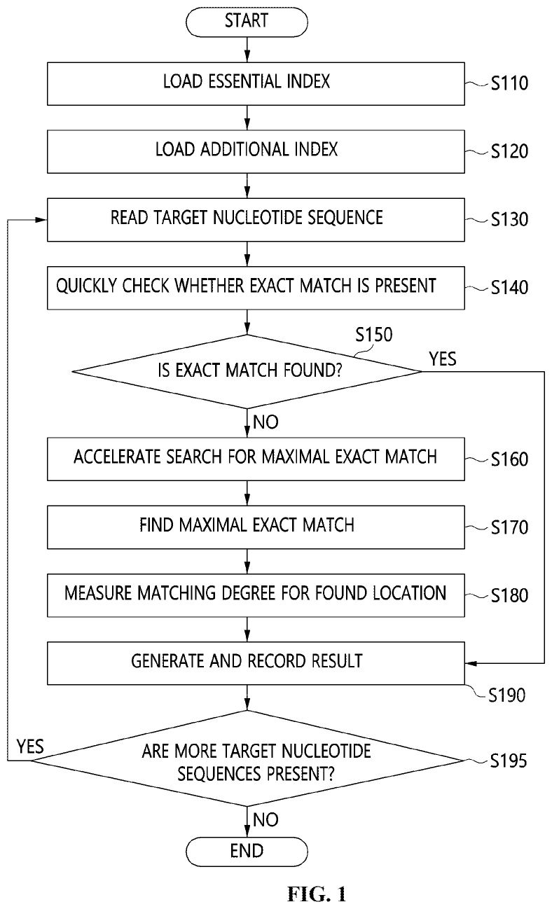

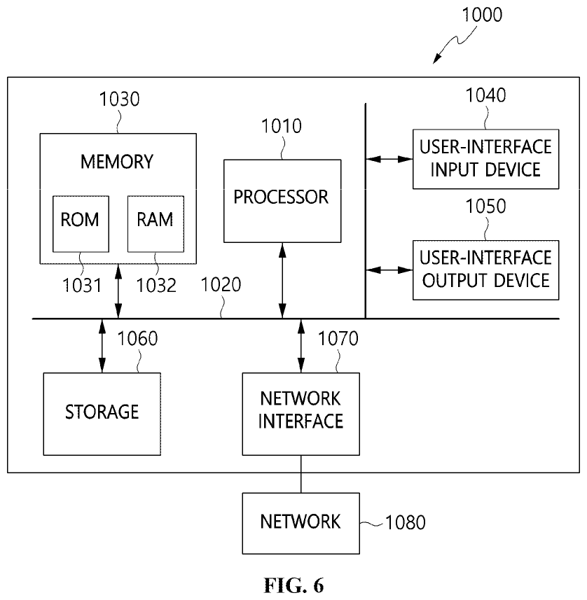

FIG. 1 is a flowchart for explaining a method for accelerating genome sequence alignment according to an embodiment. The method for accelerating genome sequence alignment may be performed by the apparatus for accelerating genome sequence alignment illustrated in FIG. 6.

Referring to FIG. 1, the method for accelerating genome sequence alignment according to an embodiment may include loading an essential index for a reference genome into memory at step S110, loading an additional index corresponding to the amount of available memory into memory at step S120, reading the target nucleotide sequence for which genome sequence alignment is to be performed at step S130, checking whether an exact match of the target nucleotide sequence is present in the reference genome based on the additional index at step S140, and generating a target nucleotide sequence alignment result using the location of the exact match of the target nucleotide sequence in the reference genome at step S190 when it is determined at step S150 that an exact match is found.

Also, the method for accelerating genome sequence alignment according to an embodiment may further include, when no exact match is found at step S150, finding a maximal exact match between the target nucleotide sequence and the reference genome based on the essential index at step S170, measuring the degree of matching between the target nucleotide sequence and the maximal exact match found in the reference genome at step S180, and generating a result representing the degree of matching at step S190. Also, finding the maximal exact match at step S180 may further include accelerating the initial step of finding the maximal exact match based on the additional index at step S160.

Here, the essential index may be used for a general genome sequence alignment process.

Here, the additional index is an index according to an embodiment, and may be added in order to improve performance by accelerating genome sequence alignment.

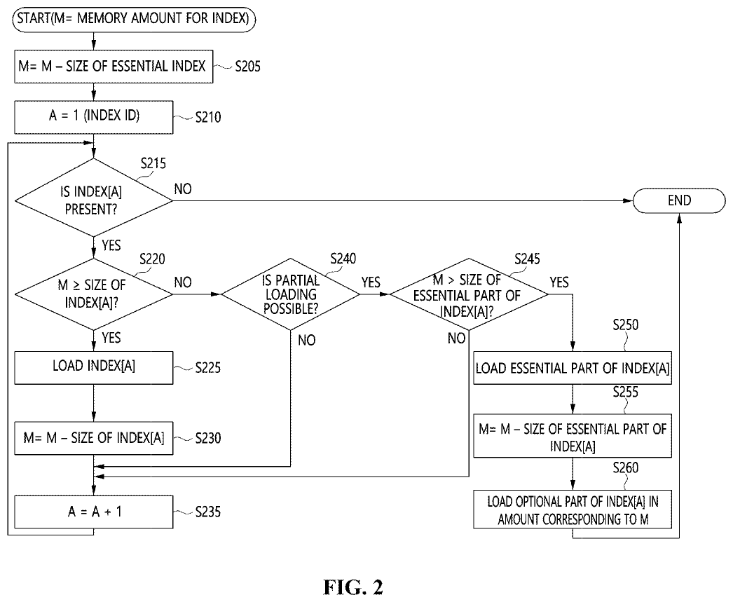

The additional index may comprise multiple additional indexes. In the case of each of the multiple indexes, the entirety thereof may be required to be loaded for an operation, or an operation may be performed even though only a part thereof is loaded. Accordingly, in an embodiment, all of the additional indexes, some of the additional indexes, or some of the additional indexes and part of a specific index may be loaded, depending on the amount of available memory in the apparatus for genome sequence alignment. Loading the additional indexes into memory at step S120 will be described in detail later with reference to FIG. 2.

The additional indexes may include a first index for quickly checking whether an exact match of the target nucleotide sequence is present.

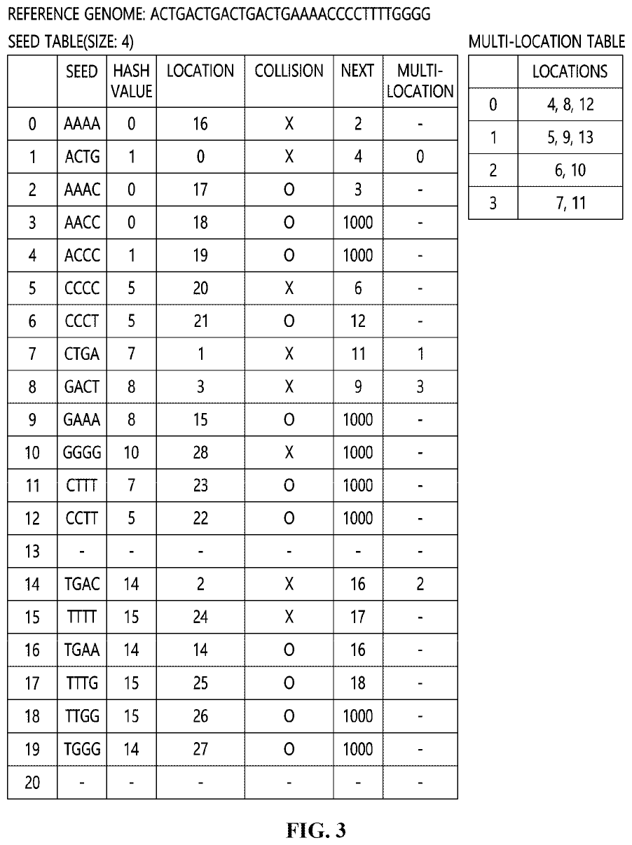

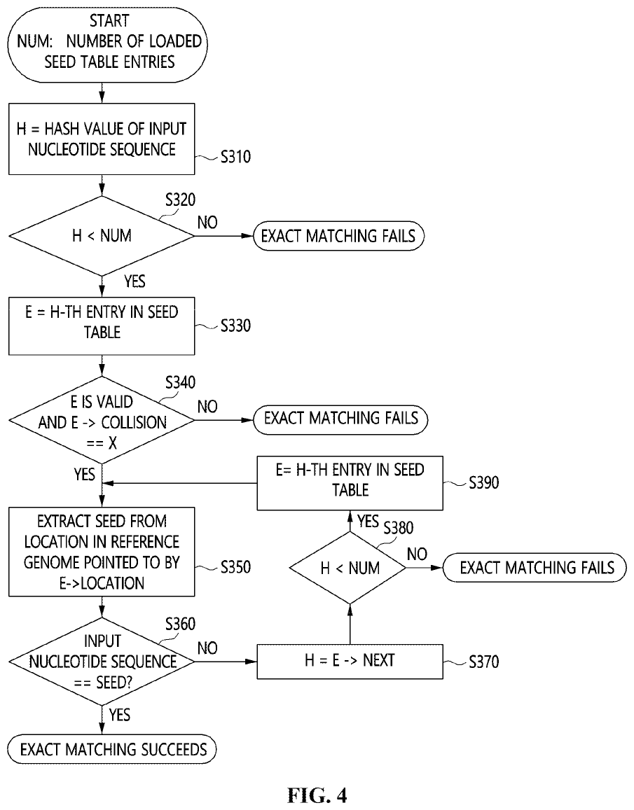

Accordingly, the first index may be used in order to quickly perform the step (S140) of checking whether an exact match of the target nucleotide sequence is present in the reference genome according to an embodiment. The configuration of the first index and checking whether an exact match is present in the reference genome using the first index at step S140 will be described in detail later with reference to FIG. 3 and FIG. 4.

Also, the additional indexes may include at least one of second indexes for accelerating a search for a maximal exact match. Accordingly, the additional indexes may be used at the step (S160) of accelerating the initial step of the search for the maximal exact match according to an embodiment.

Meanwhile, depending on the determination as to whether more input target nucleotide sequences remain at step S195, steps S130 to S190 according to an embodiment may be repeatedly performed until no more target nucleotide sequences exist.

FIG. 2 is a flowchart for explaining in detail a step of loading an additional index into memory according to an embodiment.

Referring to FIG. 2, the apparatus for accelerating genome sequence alignment calculates the amount of memory available for an additional index at step S205. If the amount of memory available for indexes to be used for genome sequence alignment is M, after step S110 described above is performed, the amount of memory available for the indexes, M, is updated by subtracting the size of the essential index from M acquired before step S110. Accordingly, M, which is the updated amount of available memory, may be used for the additional indexes.

Because an embodiment is aimed at improving the speed of genome sequence alignment depending on the amount of available memory, whether to load the additional indexes has to be decided depending on the amount of available memory calculated at step S205.

To this end, the apparatus for genome sequence alignment determines whether to sequentially load the multiple additional indexes.

Here, the order in which the additional indexes are to be loaded may be set based on the effect of each of the additional indexes on the performance of genome sequence alignment, whereby the performance of genome sequence alignment may be maximized depending on the amount of available memory in the system.

First, the apparatus for genome sequence alignment initializes a variable A, to which the ID of an additional index is assigned, to ‘1’ at step S210 and determines whether index[A] is present at step S215.

When it is determined at step S215 that index[A] is not present, the apparatus for genome sequence alignment terminates index loading.

Conversely, when it is determined at step S215 that index[A] is present, the apparatus for genome sequence alignment determines whether the amount of available memory, M, is equal to or greater than the size of index[A], which is to be loaded, at step S220.

When it is determined at step S220 that the amount of available memory, M, is equal to or greater than the size of index[A], which is to be loaded, the apparatus for genome sequence alignment loads index[A] into memory at step S225.

Then, the apparatus for genome sequence alignment updates M, which is the amount of available memory, and index[A] at steps S230 and S235 and performs step S215. That is, the size of loaded index[A] is subtracted from the previous amount of available memory, M, at step S230, whereby M is updated to the current amount of available memory. Also, index[A] is updated to the index subsequent thereto at step S235.

Meanwhile, when it is determined at step S220 that the amount of available memory, M, is less than the size of index[A] to be loaded, the apparatus for genome sequence alignment determines whether index[A] can be partially loaded at step S240.

When it is determined at step S240 that index[A] can be partially loaded, the apparatus for genome sequence alignment loads as much of index[A] as possible at steps S245 to S260.

However, when an index can be partially loaded, the index may include an essential part that is required for using the index. Accordingly, the apparatus for genome sequence alignment determines whether the amount of available memory, M, is equal to or greater than the size of the essential part of index[A] at step S245.

When it is determined at step S245 that the amount of available memory, M, is equal to or greater than the size of the essential part of index[A], the apparatus for genome sequence alignment preferentially loads the essential part of index[A] at step S250. Then, the apparatus for genome sequence alignment subtracts the size of the essential part of index[A] from the amount of available memory, that is, M, at step S255. Then, the optional part of index[A] is partially loaded in an amount corresponding to M at step S260.

Meanwhile, when it is determined at step S240 that it is impossible to partially load index[A] or when it is determined at step S245 that the amount of available memory, M, is less than the size of the essential part of index[A], the process goes to step S235, whereby the next index is considered.

Steps S215 to S260 described above may be repeatedly performed until no more additional indexes remain.

Next, a first index for quickly finding an exact match of a target nucleotide sequence and the step (S140) of quickly checking whether such an exact match is present using the first index will be described in detail with reference to FIG. 3 and FIG. 4.

FIG. 3 is an exemplary view of a first index for quickly finding an exact match of a target nucleotide sequence according to an embodiment.

Referring to FIG. 3, the first index may be configured with two tables, namely a seed table and a multi-location table.

Here, the seed table represents reference nucleotide sequences in a reference genome as hash table values using a hash function, and indices (key values) of the reference nucleotide sequences are generated in advance such that the location of a given short nucleotide sequence in the reference genome can be quickly found. Here, the unit for which an index of a nucleotide sequence is generated is called a seed.

Here, the length of a seed is the length of a target for which an exact match is to be searched for. For example, when the length of a seed is set to ‘4’, as shown in FIG. 3, different seeds corresponding to ‘4’, which is the length of the target for which an exact match is to be searched for, may be extracted from the given reference genome ‘ACTGACTGACTGACTGAAAACCCCTTTTGGGG’. For example, seeds, each of which is configured with four letters, such as ‘AAAA’, ‘ACTG’, and the like, may be extracted from the reference genome.

Such a seed table may be configured with hash values, which are acquired by applying a hash function to the respective seeds extracted from the reference genome, and hash entries.

Here, the hash function is a function applicable to the seeds extracted from the reference genome, and various embodiments therefor are possible.

Also, the hash entry may include a ‘location’ field, a ‘collision’ field, a ‘next’ field, and a ‘multi-location’ field.

Here, the ‘location’ field contains information about the location of a seed in the reference genome, and may be information about the location from which the seed starts in the reference genome when the first location in the reference genome is set to ‘0’. For example, referring to FIG. 3, because ‘ACTG’ is located at the first location in the reference genome, the value of the ‘location’ field may be ‘0’, and because ‘AAAA’ starts from the 17th location in the reference genome, the value of the ‘location’ field may be ‘16’.

The ‘collision’ field contains information about whether a hash collision occurs for the corresponding hash entry. That is, when an entry having the same hash value as the corresponding entry does not appear before the corresponding entry, the value of the ‘collision’ field may be set to ‘x’, which indicates ‘no hash collision’, whereas when an entry having the same hash value as the corresponding entry appears before the corresponding entry, the value of the ‘collision’ field may be set to ‘o’, which indicates ‘hash collision’. For example, referring to FIG. 3, because the hash value of ‘AAAA’ is 0 and because there is no seed having the same hash value as ‘AAAA’ before that, the value of the ‘collision’ field is set to ‘x’. However, in the case of ‘AAAC’, because the hash value thereof is 0 and because the seed ‘AAAA’ having the same hash value as ‘AAAC’ is located before that, the value of the ‘collision’ field is set to ‘o’.

Also, the ‘next’ field indicates the index number of the next entry having the same hash value.

Here, if N seeds have the same hash value, each of the first to (N−1)-th entries has the index number of the next entry thereof as the value of the ‘next’ field. For example, referring to FIG. 3, because the next entry having the same hash value as ‘AAAA’ (here, the hash value is ‘0’) corresponds to the seed ‘AAAC’ having an entry index number of 2, the value of the ‘next’ field of the entry corresponding to the seed ‘AAAA’ is set to ‘2’.

Also, the N-th entry, among the N seeds having the same hash value, or an entry, the hash value of which is not equal to any of the hash values of the other entries, has a value greater than the total number of entries in the hash table as the value of the ‘next’ field. For example, the value of the ‘next’ field for the seed ‘AAAC’, which is the last entry having a hash value of ‘0’, may be set to ‘1000’, which is greater than the total number of entries in the hash table. Also, because the entry having a hash value of ‘10’ is only the seed ‘GGGG’, the value of the ‘next’ field therefor may be set to ‘1000’, which is greater than the total number of entries in the hash table.

Also, the ‘multi-location’ field indicates, when the same seed is found at two or more locations in the reference genome, an index in the multi-location table at which the corresponding locations are recorded.

Meanwhile, when a single seed is found at two or more locations in the reference genome, the multi-location table records the corresponding locations all together. Here, the location included in the seed table is not recorded.

For example, referring to FIG. 3, the seed ‘ACTG’ is found at four locations in the reference genome, and the locations may be 0, 4, 8, and 12. Accordingly, the locations excluding the first location, that is, ‘4, 8, 12’, may be recorded in the entry having an index number ‘0’ in the multi-location table. Also, in the seed table, the hash entry corresponding to the first location of the seed ‘ACTG’ may have the index number in the multi-location table as the value of the ‘multi-location’ field.

Meanwhile, in the seed table of FIG. 3, the index numbers and the seeds, that is, the fields denoted by reference numeral 11, are not actually stored. This is because the seeds may be extracted by reading the reference genome when location information is given and because the hash values thereof may also be calculated.

Also, in the index for quickly checking whether an exact match of a target nucleotide sequence is present, the seed table may be used even though only a portion thereof is loaded. However, the multi-location table may be used only when the entirety thereof is loaded. This is because entries at various locations in the seed table refer to the entries in the multi-location table.

FIG. 4 is a flowchart for explaining a step of quickly checking whether an exact match of a target nucleotide sequence is present according to an embodiment.

Here, an embodiment in which an exact match is searched for when a first index for quickly finding the exact match is partially loaded is illustrated. However, the operation in FIG. 4 may be applied in the same manner even when the entirety of the first index is loaded.

The apparatus for genome sequence alignment calculates the hash value of an input target nucleotide sequence at step S310.

Subsequently, the apparatus for genome sequence alignment determines whether the calculated hash value is less than NUM, which is the number of loaded entries, among the entries of a seed table, at step S320.

When it is determined at step S320 that the calculated hash value is not less than NUM, the apparatus for genome sequence alignment determines that an exact match could not be quickly found, and then performs step S160. That is, even if an exact match is not found using a quick search, the input target nucleotide sequence may be aligned using the existing genome sequence alignment method that uses only the essential index.

Conversely, when it is determined at step S320 that the calculated hash value is less than NUM, the apparatus for genome sequence alignment searches for a hash entry, the index number of which in the seed table corresponds to the hash value, at step S330.

Then, the apparatus for genome sequence alignment determines whether an entry, the index number of which in the seed table corresponds to the hash value, is found and whether the value of the ‘collision’ field thereof is ‘x’ at step S340.

When it is determined at step S340 that an entry, the index number of which in the seed table corresponds to the hash value, is not found or that the value of the ‘collision’ field of the found entry is ‘o’, the apparatus for genome sequence alignment determines that the attempt to quickly find an exact match has failed, and then performs step S160.

That is, the actual hash value of the found entry may be different from the hash value of the input target nucleotide sequence. Also, an entry having the same hash value as the target nucleotide sequence may not be present, or the found hash entry may not be valid.

Conversely, when it is determined at step S340 that an entry, the index number of which in the seed table corresponds to the hash value, is found and when the value of the ‘collision’ field of the found entry is ‘x’, the apparatus for genome sequence alignment extracts a seed from the reference genome using the location information stored in the found entry at step S350.

Subsequently, the apparatus for genome sequence alignment checks whether the extracted seed matches the target nucleotide sequence at step S360.

When it is determined at step S360 that the extracted seed matches the target nucleotide sequence, it is determined that exact matching succeeds.

Conversely, when it is determined at step S360 that the extracted seed does not match the target nucleotide sequence, the apparatus for genome sequence alignment searches for the entry corresponding to the value of the ‘next’ field of the hash entry in the seed table at steps S370 to S390, and performs S350 so as to check whether the found seed matches the target nucleotide sequence.

Here, the apparatus for genome sequence alignment determines whether the value of the ‘next’ field is less than NUM at step S380, thereby determining whether the entry corresponding thereto is loaded. When it is determined at step S380 that the value of the ‘next’ field is not less than NUM, it is determined that the entry corresponding thereto is not loaded, and the apparatus for genome sequence alignment determines that exact matching fails.

Meanwhile, when it is determined that exacting matching succeeds, the apparatus for genome sequence alignment checks the value of the ‘multi-location’ field of the entry corresponding to the seed that exactly matches the target nucleotide sequence. When the value of the ‘multi-location’ field of the entry is present, all of the exact matches of the input target nucleotide sequence may be found at the locations in the reference genome that are collected as the value of the ‘location’ field of the multi-location table.

Through the above-described process, the apparatus for genome sequence alignment may quickly determine whether an exact match of each input nucleotide sequence is present using only part of the index.

Also, in an embodiment, when a part of the seed table is selected, entries, the hash values of which is equal to or less than a specific value, are selected.

This method is effective because the locations of nucleotide sequences extracted by a sequencing machine are randomly distributed across the entire genome. Also, when a commonly used hash function is used, seeds are evenly distributed over a hash table. Accordingly, when part of the seed table is loaded from the beginning so as to have a size of 10% of the seed table, about 10% of the exact matches of the input nucleotide sequence may be found.

Meanwhile, the second additional index may be an index used for the step (S160) of accelerating a search for the maximal exact match illustrated in FIG. 2.

The Burrows-Wheeler Transform (BWT) algorithm and the Ferragina Manzini (FM) index structure, which are commonly used to find a maximal exact match in genome sequence alignment, are configured such that the locations in a long string at which the first character of a short string is located are searched for, and among the found locations, locations at which the first character of the short string is followed by the second character thereof are searched for.

Because there are four types of nucleotides, the number of possible nucleotide sequences exponentially increases depending on the length thereof, but when the length is short, the number of possible nucleotide sequences is small. Using this fact, an index for storing a result value for the initial step of the BWT algorithm may be formed and used.

Table 1 below indicates the size of an index for acceleration of a search fora maximal exact match.

TABLE 1

| | num of | storing all ranges | | | storing final range | | |
|---|---|---|---|---|---|---|---|
| length | cases | entry size | index size | | entry size | index size | |
| 10 | 1.05M | 112 B | 0.12 | GB | 16 B | 0.02 | GB |
| 11 | 4.19M | 124 B | 0.50 | GB | 16 B | 0.06 | GB |
| 12 | 16.78M | 136 B | 3 | GB | 16 B | 0.25 | GB |
| 13 | 67.11M | 148 B | 12 | GB | 16 B | 1 | GB |
| 14 | 268.44M | 160 B | 48 | GB | 16 B | 4 | GB |
| 15 | 1073M | 172 B | 192 | GB | 16 B | 16 | GB |
| 16 | 4294M | 184 B | 768 | GB | 16 B | 64 | GB |

In an embodiment, 12 bytes are required to store a range for a single length. Then, four bytes are added in order to store the extendable maximum length for each entry. The index size is set on the assumption that each entry is aligned in units of 64 bytes, which is a block unit of a CPU cache.

Table 1 illustrates the number of possible cases depending on each nucleotide sequence length and the capacity required for storing the result values of the BWT algorithm according to an embodiment. Two methods are used depending on the actual implementation of the BWT algorithm.

First, ‘storing all ranges’ is storing all result values for the respective lengths. For example, when results for a length of 10 are stored, the result values of the BWT algorithm for all of the respective lengths from 1 to 10 are stored. On the other hand, ‘storing final range’ is storing only the result values of the BWT algorithm only for a length of 10. That is, only maximal exact matches of a length of 10 between the target nucleotide sequence and the reference genome are stored.

According to Table 1, the result values of the BWT algorithm for a nucleotide sequence having a length of 10 to 15 may be stored using only the capacity of several gigabytes to tens of gigabytes.

The second index for accelerating a search for a maximal exact match according to an embodiment generates and stores all of the possible sequences for a given length. Accordingly, when only a part of the second index is loaded, whether the range of the nucleotide sequence to be used is included in the loaded second index is checked, and the second index is used only when the range is included therein.

According to the above-described embodiment, the following effects may be obtained.

First, performance may be improved depending on a memory size, and remaining memory may be used.

Second, exact matching may be quickly determined.

Third, ‘90%’ mentioned in the description of the related art is a figure when an error in a sequencing machine is not considered, and when a nucleotide sequence having a length of 148 is given as actual data, about 65˜76% thereof completely matches a reference genome.

In an embodiment, as described above, two additional indexes through which genome sequence alignment can be accelerated are proposed, and a method of improving the performance of genome sequence alignment based on the additional indexes depending on the amount of available memory in the system is proposed.

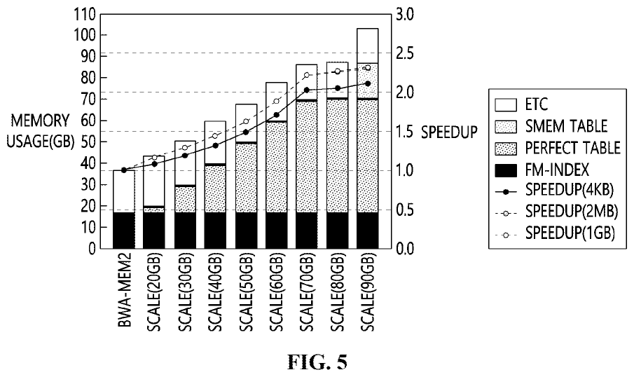

FIG. 5 is an experimental result of implementation of an embodiment in BWA-MEM2, which is a genome sequence alignment program.

Referring to FIG. 5, ‘SCALE’ represents various embodiments according to the present invention, and the number in paratheses indicates the capacity of memory available for an index. ‘Speedup’ is represented for each case in which a 4 kB page (default), a 2 MB page, or a 1 GB page is used for indexes. ‘FM-Index’ indicates an essential index, and ‘Perfect table’ and ‘SMEM table’ indicate additional indexes. The ‘ETC’ area indicates the amount of memory required for execution of a program, excluding the amount of memory for the indexes, and is not included in the memory limit for the indexes.

Both an index (perfect table) for quickly finding an exact match and two indexes (commonly called SEMM table) for accelerating a search for a maximal exact match are applied. The implementation is made such that all or part of the perfect table can be loaded, but in the case of the SEMM table, loading only of the entirety thereof is allowed because the performance improvement effect of application thereof is not great. Also, the order in which the additional indexes are loaded is set based on the performance improvement effect for every 1 gigabyte. As a result, the indexes are used in the order of SEMM table for storing all ranges (length: 11)→perfect table→SEMM table for storing a final range (length: 15). Also, NCBI SRA: SRX206890 is used as the input nucleotide sequence.

The result shows that, when the memory capacity for indexes increases from 20 GB to 90 GB, even though a 4 kB page, which is a default page size of a system, is used, a performance improvement of up to 2.1 times is obtained. Particularly, it can be seen that an almost linear performance improvement is obtained in the section from 20 GB to 70 GB, in which the perfect table is partially loaded.

FIG. 6 is a view illustrating a computer system configuration according to an embodiment.

The apparatus for accelerating genome sequence alignment according to an embodiment may be implemented in a computer system 1000 including a computer-readable recording medium.

The computer system 1000 may include one or more processors 1010, memory 1030, a user-interface input device 1040, a user-interface output device 1050, and storage 1060, which communicate with each other via a bus 1020. Also, the computer system 1000 may further include a network interface 1070 connected to a network 1080. The processor 1010 may be a central processing unit or a semiconductor device for executing a program or processing instructions stored in the memory 1030 or the storage 1060.

The program may perform the above-described method for accelerating genome sequence alignment.

The memory 1030 and the storage 1060 may be storage media including at least one of a volatile medium, a nonvolatile medium, a detachable medium, a non-detachable medium, a communication medium, and an information delivery medium. For example, the memory 1030 may include ROM 1031 or RAM 1032.

According to the disclosed embodiment, some or all of the acceleration methods are used depending on the available memory capacity, whereby the speed of genome sequence alignment may be improved in proportion to the available memory capacity.

According to the disclosed embodiment, the speed of genome sequence alignment may be improved using available memory in a system without special hardware.

According to the disclosed embodiment, the performance of genome sequence alignment is improved using the high match rate between genomes, and the speed thereof may be improved compared to a search for an exact match using the existing BWT algorithm.

Although embodiments of the present invention have been described with reference to the accompanying drawings, those skilled in the art will appreciate that the present invention may be practiced in other specific forms without changing the technical spirit or essential features of the present invention. Therefore, the embodiments described above are illustrative in all aspects and should not be understood as limiting the present invention.

What is claimed is:

1. An apparatus for accelerating genome sequence alignment, comprising:
memory in which at least one program is recorded; and
a processor for executing the program,
wherein the program performs
loading an essential index for a reference genome into memory;
loading an additional index corresponding to an amount of available memory into memory;
reading a target nucleotide sequence for which genome sequence alignment is to be performed;
checking whether an exact match of the target nucleotide sequence is present in the reference genome based on the additional index; and
generating a result of alignment of the target nucleotide sequence using a location of the exact match in the reference genome when the exact match is found.

2. The apparatus of claim 1, wherein, when loading the additional index into memory, the program uses available memory, an amount of which is calculated by subtracting a size of the essential index from a total amount of memory to be used for indexes for genome sequence alignment, in order to load the additional index.

3. The apparatus of claim 2, wherein:
when loading the additional index into memory, if the additional index comprises two or more additional indexes, the program sequentially loads the additional indexes, and
an order in which the additional indexes are loaded is determined based on an effect of each of the additional indexes on genome sequence alignment performance.

4. The apparatus of claim 2, wherein:
when loading the additional index into memory, the program loads all or part of the additional index depending on whether the amount of available memory is equal to or greater than a size of the additional index to be loaded, and
when part of the additional index is loaded, the program preferentially loads an essential part of the additional index.

5. The apparatus of claim 1, wherein:
the additional index includes a first index that is used when checking whether the exact match of the target nucleotide sequence is present in the reference genome is performed, and
the first index includes a seed table configured with hash entries corresponding to respective seeds having a predetermined length, which are extracted from the reference genome, and a multi-location table in which two or more locations of an identical seed in the reference genome are collectively mapped to a single index.

6. The apparatus of claim 5, wherein the hash entry includes information about a location of a seed in the reference genome, information about whether the hash entry has a hash collision, an index number of a next hash entry having a same hash value as the hash entry, and information about an index in the multi-location table.

7. The apparatus of claim 6, wherein, when checking whether the exact match of the target nucleotide sequence is present in the reference genome based on the additional index, the program performs
calculating a hash value of the target nucleotide sequence;
searching for a hash entry corresponding to the hash value when the hash value is less than a number of loaded hash entries of the seed table;
when the hash entry corresponding to the hash value is found and when the found entry is not an entry having a hash collision, extracting a seed from the reference genome using location information stored in the found entry;
checking whether the extracted seed matches the target nucleotide sequence; and
when the extracted seed is determined to match the target nucleotide sequence, searching the multi-location table for all exact matches of the target nucleotide sequence in the reference genome.

8. The apparatus of claim 7, wherein, when checking whether the extracted seed matches the target nucleotide sequence is performed, if it is determined that the extracted seed does not match the target nucleotide sequence, the program searches for an entry corresponding to a next value of the hash entry in the seed table and further performs checking whether a seed of the found entry matches the target nucleotide sequence.

9. The apparatus of claim 1, wherein:
when the exact match of the target nucleotide sequence is not found in the reference genome, the program performs
finding a maximal exact match between the target nucleotide sequence and the reference genome based on the essential index;
measuring a degree of matching between the target nucleotide sequence and the maximal exact match found in the reference genome; and
generating a result indicating the degree of matching, and
when finding the maximal exact match is performed, the program accelerates an initial step of finding the maximal exact match based on a second index of the additional index.

10. A method for accelerating genome sequence alignment, comprising:
loading an essential index for a reference genome into memory;
loading an additional index corresponding to an amount of available memory into memory;
reading a target nucleotide sequence for which genome sequence alignment is to be performed;
checking whether an exact match of the target nucleotide sequence is present in the reference genome based on the additional index; and
generating a result of alignment of the target nucleotide sequence using a location of the exact match in the reference genome when the exact match is found.

11. The method of claim 10, wherein
loading the additional index into memory comprises loading all or part of the additional index depending on whether the amount of available memory is equal to or greater than a size of the additional index to be loaded, and
when part of the additional index is loaded, an essential part of the additional index is preferentially loaded.

12. The method of claim 10, wherein:
the additional index includes a first index that is used when checking whether the exact match of the target nucleotide sequence is present in the reference genome is performed, and
the first index includes a seed table configured with hash entries corresponding to respective seeds having a predetermined length, which are extracted from the reference genome, and a multi-location table in which two or more locations of an identical seed in the reference genome are collectively mapped to a single index.

13. The method of claim 12, wherein the hash entry includes information about a location of a seed in the reference genome, information about whether the hash entry has a hash collision, an index number of a next hash entry having a same hash value as the hash entry, and information about an index in the multi-location table.

14. The method of claim 13, wherein checking whether the exact match of the target nucleotide sequence is present in the reference genome based on the additional index includes
calculating a hash value of the target nucleotide sequence;
searching for a hash entry corresponding to the hash value when the hash value is less than a number of loaded hash entries of the seed table;
when the hash entry corresponding to the hash value is found and when the found entry is not an entry having a hash collision, extracting a seed from the reference genome using location information stored in the found entry;
checking whether the extracted seed matches the target nucleotide sequence; and
when the extracted seed is determined to match the target nucleotide sequence, searching the multi-location table for all exact matches of the target nucleotide sequence in the reference genome.

15. The method of claim 14, further comprising:
when it is determined that the extracted seed does not match the target nucleotide sequence as a result of checking whether the extracted seed matches the target nucleotide sequence,
searching for an entry corresponding to a next value of the hash entry in the seed table and checking whether a seed of the found entry matches the target nucleotide sequence.

16. The method of claim 10, further comprising:
when the exact match of the target nucleotide sequence is not found in the reference genome,
finding a maximal exact match between the target nucleotide sequence and the reference genome based on the essential index;
measuring a degree of matching between the target nucleotide sequence and the maximal exact match found in the reference genome; and
generating a result indicating the degree of matching,
wherein, when finding the maximal exact match is performed, an initial step of finding the maximal exact match is accelerated based on a second index of the additional index.

17. A method for accelerating genome sequence alignment, comprising:
loading an essential index for a reference genome into memory;
loading an additional index corresponding to an amount of available memory into memory;
reading a target nucleotide sequence for which genome sequence alignment is to be performed;
checking whether an exact match of the target nucleotide sequence is present in the reference genome based on a first index of the additional index;
generating a result of alignment of the target nucleotide sequence using a location of the exact match in the reference genome when the exact match is found;
finding a maximal exact match between the target nucleotide sequence and the reference genome based on the essential index when the exact match of the target nucleotide sequence is not found;
measuring a degree of matching between the target nucleotide sequence and the maximal exact match found in the reference genome; and
generating a result indicating the degree of matching,
wherein when finding the maximal exact match is performed, an initial step of finding the maximal exact match is accelerated based on a second index of the additional index.

18. The method of claim 17, wherein:
the first index includes a seed table configured with hash entries corresponding to respective seeds having a predetermined length, which are extracted from the reference genome, and a multi-location table in which two or more locations of an identical seed in the reference genome are collectively mapped to a single index, and
the hash entry includes information about a location of a seed in the reference genome, information about whether the hash entry has a hash collision, an index number of a next hash entry having a same hash value as the hash entry, and information about an index in the multi-location table.

19. The method of claim 18, wherein checking whether the exact match of the target nucleotide sequence is present in the reference genome based on the first index includes
calculating a hash value of the target nucleotide sequence;
searching for a hash entry corresponding to the hash value when the hash value is less than a number of loaded hash entries of the seed table;
when the hash entry corresponding to the hash value is found and when the found entry is not an entry having a hash collision, extracting a seed from the reference genome using location information stored in the found entry;
checking whether the extracted seed matches the target nucleotide sequence; and
when the extracted seed is determined to match the target nucleotide sequence, searching the multi-location table for all exact matches of the target nucleotide sequence in the reference genome.

20. The method of claim 19, further comprising:
when it is determined that the extracted seed does not match the target nucleotide sequence as a result of checking whether the extracted seed matches the target nucleotide sequence,
searching for an entry corresponding to a next value of the hash entry in the seed table, and checking whether a seed of the found entry matches the target nucleotide sequence.
